(19) United States
(12) Patent Application Publication
(10) Pub. No.: US 2004/0116668 A1
(43) Pub. Date: Jun. 17, 2004

(54) Zis-sr nucleic acid and amino acid sequences involved in the regulated secretory pathway and/or the regulation of the neuroendocrine phenotype (nep)

(76) Inventor: Robert Day, Sherbrooke (CA)

Correspondence Address: QUARLES & BRADY LLP, 411 E. WISCONSIN AVENUE, SUITE 2040, MILWAUKEE, WI, 53202-4497, US

(21) Appl. No.: 10/470,283

(22) PCT Filed: Jan. 29, 2002
(86) PCT No.: PCT/CA02/00101

(51) Int. Cl. C07K 14/47; A61K 38/17; C07H 21/04

(52) U.S. Cl. 530/350; 514/12; 536/23.5; 435/69.1; 435/320.1; 435/325

(57) ABSTRACT

The present invention relates to genes and protein encoded thereby that regulate the secretory pathway and/or the neuroendocrine phenotype (NEP) in cells and method of isolating same. More particularly, the present invention relates to long-term therapies for diseases or conditions associated with a loss function. More particularly, the present invention relates to the treatment of such diseases using a cell replacement therapy. In particular, the invention relates to genes involved in cellular differentiation and genes that modulate the formation of the regulated secretory pathway. The invention thus also concerns a method to identify such genes, the genes, variants or fragments thereof, vectors comprising same, the products of these genes, variants or fragments thereof and to cells expressing same. In a particular embodiment, the invention relates to the characterization of Zis-SR a novel sequence involved in the secretory pathway in cells.

### FIELD OF THE INVENTION

The present invention relates to genes and protein encoded thereby that are involved in the regulated secretory pathway and/or regulate the neuroendocrine phenotype (NEP) and method of isolating same. More particularly, the invention relates to Zis-SR sequences. The invention also relates to therapies for diseases or conditions associated with a loss of function of factors involved in the secretory pathway and more particularly of Zis-SR.

### BACKGROUND OF THE INVENTION

The development of strategies to repair loss of function of endocrine cells, for example as in diabetes, or loss of neurons, for example as in degenerative diseases such as Parkinson's disease or Alzheimer's disease, is a major ongoing therapeutic challenge. Current strategies for treatment utilize replacement therapies that provide replacement of only certain functions carried out by the lost cells. For example in diabetes, the major loss of function is caused by a lack of insulin, an important hormone that controls glucose metabolism. In diabetes, the lack of endogenous insulin is compensated by insulin injections that helps reduce blood glucose levels and thus prevents the unwanted effects of hyperglycemia. In the case of Parkinson's disease, a major result of the degeneration of striato-nigral neurons is the lack of dopamine, an important neurotransmitter that is utilized within this context to control motor movements. Once again, therapy for Parkinson's disease involves providing medication that compensates for the lack of dopamine.

The present therapies have significant limitations and certainly do not provide a long-term cure. In the example of diabetes, while insulin replacement therapy has saved many lives, it has major problems since insulin levels fluctuate significantly depending on how the insulin administration is carried out. Unregulated insulin delivery results in peaks and valleys of insulin that have serious repercussions. In the case of Parkinson's disease, replacing dopamine has only short-term benefits, as the system tends to adapt to increased dopamine levels through receptor desensitization. Furthermore, dopamine by itself cannot compensate for all the other neurotransmitters found within the striato-nigral neurons, for example such as dynorphin or substance P, which are recognized for also having an impact in the co-ordination of motor movements. It would be quite difficult to replace all the neurotransmitters or hormones found within one cell by simply administering all of these in a drug cocktail in order to achieve the correct temporal regulatory requirements. Thus a new strategy is required.

In order to solve these problems and also to provide a long-term cure, a better therapeutic strategy would be to provide replacement cells. Cell replacement therapies should provide a long-term solution as well as additional benefits knowing that the cell used for replacement therapy would contain all necessary elements required for all cellular functions. In order to produce such cells. It is important to identify critical genes in the process of neuroendocrine cellular differentiation.

There thus remains a need to provide a new strategy to treat loss of function of cells and more particularly, loss of function of endocrine cells, or loss of neurons. There also remains a need to improve the current replacement therapies.

There also remains a need to improve the long-term therapies to treat diseases or conditions associated with loss of function.

There also remains a need to provide cell replacement therapies and to identify critical genes involved in the process of neuroendocrine cellular differentation. These genes would then be used to fashion the neural or endocrine cell required for cellular replacement therapy. At this time, genes controlling neuroendocrine differentiation have not been identified, and model systems need to be developed in order to carry out this identification.

The present invention seeks to meet these and other needs.

The present description refers to a number of documents, the content of which is herein incorporated by reference in their entirety.

### SUMMARY OF THE INVENTION

The present invention concerns the regulated secretory pathway and to cells in which this process occurs.

In one embodiment, the invention provides a method to modulate the differentiation of cells and/or to modulate the regulated secretory pathway.

Genes that control cellular differentiation are being actively sought. However, appropriate methods to achieve this goal are highly variable. Simple cell models are required to permit the identification of specific genes that control the differentiation of embryonic stem cells into neurons or endocrine cells.

In accordance with one embodiment, there is described a cell line that specifically and rapidly differentiates into a neuroendocrine cell and subsequent to this differentiation event, the genes involved in this process can be identified. Once identified, these genes can serve the purpose to develop replacement neurons to be used in neurodegenerative diseases such as Parkinson's or Alzheimer's disease. These genes could also serve to produce replacement beta-cells in diabetes.

The present invention concerns a new strategy to treat loss of function of cells and more particularly, loss of function of endocrine cells, or loss of neurons.

In addition, the invention relates to improved therapies to treat diseases or conditions associated with loss of function.

Also, the invention relates to a method to modulate the differentiation of cells and/or to modulate the regulated secretory pathway.

Further, the invention relates to cell replacement therapies and to identify critical genes involved in the process of neuroendocrine cellular differentiation.

In a particular embodiment, the invention relates to the identification of a novel gene involved in the secretory pathway and neuroendocrine cellular differentiation. Using the method of the present invention, Zis-SR has been identified and characterized. In accordance with the present invention, it is shown that Zis-SR and the modulation thereof can modulate the secretory pathway in cells.

The present invention identifies Zis-SR as a protein having a previously unknown extended SR region.

Prior to the present invention, a role of Zis-SR or related sequences in the secretory pathway in cells had not been taught. The present invention therefore opens the way to the modulation of the secretory pathway in cells using Zis-SR, Zis-SR-interacting factors or molecules or agents interfering with Zis-SR or with substrates or targets thereof.

The present invention further opens the way to the design, testing and identification of agents which modulate the secretory pathway in cells using screening assays.

The present invention also relates to the treatment of such diseases using a cell replacement therapy. In particular, the invention relates to genes involved in cellular differentiation and genes that modulate the formation of the regulated secretory pathway. The invention thus also concerns a method to identify such genes, the genes, variants or fragments thereof, vectors comprising same, the products of these genes, variants or fragments thereof and to cells expressing same.

In order to provide a clear and consistent understanding of terms used in the present description, a number of definitions are provided hereinbelow.

The terminology “SR extended region of Zis-SR” is meant to relate to the extension identified herein as starting from the Histidine residue in the mouse Zis-SR sequence at position 243. A non-limiting example of this SR extension is a 68 amino acid extension in mZis-SR. Of course, while the extension is based on the numbering from the mouse sequence, the SR extended region is not limited to mouse Zis-SR.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA, RNA or chimeras thereof) for practicing the present invention may be obtained according to well known methods.

As used herein, the term “physiologically relevant” is meant to describe interactions which can modulate transcription of a gene in its natural setting.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence refers to a molecule comprised generally of the deoxyribonucleotides adenine (A), guanine (G), thymine (T) and/or cytosine (C), often in a double-stranded form, which can comprise or include a “regulatory element”. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA), deoxyribonucleic acid (DNA), or chimeras thereof.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods. While they are usually in a single-stranded form, they can be in a double-stranded form and even contain a “regulatory region”.

As used herein, a “primer” defines an oligonucleotide which is capable of annealing to a target sequence, thereby creating a double stranded region which can serve as an initiation point for DNA synthesis degradation or other known suitable purposes.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). In a particular embodiment, amplification is carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used, for example, for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCA™ boxes. Prokaryotic promoters contain −10 and −35 consensus sequences, which serve to initiate transcription and the transcript products contain Shine-Dalgarno sequences, which serve as ribosome binding sequences during translation initiation.

As used herein, the designation “functional derivative” denotes, in the context of a functional derivative of a sequence whether a nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

In a particular embodiment, the functional derivative of Zis-SR is a non-mammalian, or lower eukaryote homolog.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, uagent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of at least one interacting domain of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a defect in the regulated secretory pathway. Alternatively, the molecules identified in accordance with the teachings of the present invention find utility in the development of more efficient compounds that affect the secretory pathway or neuroendocrine phenotype.

Alternatively, an indicator cell in accordance with the present invention can be used to identify antagonists or agonists of the secretory pathway. For example, the test molecule or molecules are incubated with the host cell and secretion assessed directly or indirectly. Methods of assessing at the qualitative and/or quantitative level the regulated secretory pathway are known in the art and non-limiting examples thereof are exemplified herein. In one embodiment, an indication and relative strength of the antagonistic properties of the molecule(s) can be provided by comparing the level of gene expression in the indicator cell in the presence of the agonist, in the absence of test molecules v. in the presence thereof. Of course, the antagonistic effect of a molecule can also be determined in the absence of agonist, simply by comparing the level of expression of the reporter gene product in the presence and absence of the test molecule(s).

It shall be understood that the “in vivo” experimental model can also be used to carry out an “in vitro” assay. For example, cellular extracts from the indicator cells can be prepared and used in an “in vitro” test.

As used herein the recitation “indicator cells” refers, for example, to cells that express at least a portion of Zis-SR which is biologically active in the secretory pathway. In another embodiment, the indicator cell can express a Zis-SR which is mutated and has a defect in cell secretion. Such an indicator cell can be used to screen for molecules which can restore secretion in the indicator cell. In certain embodiments, the indicator cells have been engineered so as to express a chosen derivative, fragment, homolog, or mutant of Zis-SR. Non-limiting examples of such mutants or derivatives include mutants in the extended SR region of Zis-SR. In one particular embodiment, these mutants include an assessment of the role of the phosphorylation of this extended SR region on the functioning of the secretory pathway in cells.

In one embodiment, a domain of one of the Zis-SR of the present invention may be provided as a fusion protein. The design of constructs therefor and the expression and production of fusion proteins are well known in the art (Sambrook et al., 1989, supra; and Ausubel et al., 1994, supra).

Non limiting examples of such fusion proteins include hemaglutinin fusions and GluthioneS-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art (from bacteria to human). Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) Zis-SR protein. It will be clear to the person of ordinary skill that whether an interaction domain of the present invention, variant, derivative, or fragment thereof retains its function in secretion can be readily determined by using the teachings and assays of the present invention and the general teachings of the art.

As known in the art, the proteins and nucleic acids of the present invention can be modified, for example by in vitro mutagenesis, to dissect the structure-function relationship thereof and permit a better design and identification of modulating compounds. However, some derivative or analogs having lost their biological function may still find utility, for example for raising antibodies, identifying agonists, or acting as dominant negative mutants. Such analogs or derivatives could be used for example to raise antibodies to the extended SR domain of the Zis-SR of the present invention. These antibodies could be used for detection or purification purposes. In addition, these antibodies could also act as competitive or non-competitive inhibitor and be found to be modulators of the regulated secretory pathway.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra).

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art. Design and testing of antisense sequences are exemplified hereinbelow using the mouse sequence. In one embodiment, the antisense targets the extended SR region of human Zis-SR. A person of ordinary skill can design such antisense sequences using well-known methods.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto. In one particularly preferred embodiment of the present invention, the antibody of the present invention, polyclonal, monoclonal, humanized or the like, is directed specifically to the extended SR region of Zis-SR.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, cells having a defect in secretion can be isolated from the afflicted individual, transformed with a DNA construct according to the invention and reintroduced to the afflicted individual in a number of ways. Alternatively, the DNA construct can be administered directly to the afflicted individual. The DNA construct can also be delivered through a vehicle such as a liposome, which can be designed to be targeted to a specific cell type, and engineered to be administered through different routes.

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, nucleic acid, and molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

Yet in another embodiment, the present invention relates to an assay to screen for drugs for the treatment of a defect associated with the secretory pathway in cells. In a particular embodiment, such assays can be designed using cells from patients having a known defect in secretion.

Also provided within the present invention is a compound having therapeutic effect on the secretory pathway in cells by a method comprising: providing a screening assay comprising a measurable biological activity of a Zis-SR protein or gene of the present invention; contacting the screening assay with a test compound; and detecting if the test compound modulates the biological activity of a Zis-SR protein or gene, wherein a test compound which modulates the biological activity is a compound with this therapeutic effect.

While the present invention is exemplified with mouse Zis-SR (nucleic acid and proteins), the invention should not be so limited. Indeed, in view of the significant conservation of this gene throughout evolution, and as shown in FIGS. 5, 6 and 8, sequences from different species could be used in the assays of the present invention. Non-limiting examples are rat and human Zis-RS ortholog gene which show very high identity with the mouse Zis-SR gene. In one particular embodiment, the Zis-SR sequences are from a mammalian species. In other embodiments, the Zis-SR sequences are from non-mammalian species. Chimeras of Zis-SR sequences are also within the scope of the present invention.

As used herein, “Zis-SR biological activity” refers to any detectable biological activity of Zis-SR gene or protein. This includes any physiological function attributable to a Zis-SR gene or protein. It can include the specific biological activity of Zis SR proteins in secretion. Zis-SR biological activity is not limited, however, to these most important biological activities herein identified. Biological activities may also include simple binding or pKa analysis of Zis-SR with compounds, substrates, interacting proteins, and the like. For example, by measuring the effect of a test compound on its ability to increase or inhibit such Zis-SR binding or interaction is measuring a biological activity of Zis-SR according to this invention. Zis-SR biological activity includes any standard biochemical measurement of Zis-SR such as conformational changes, phosphorylation status or any other feature of the protein that can be measured with techniques known in the art. Finally, Zis-SR biological activity also includes activities related to Zis-SR gene transcription or translation, or any biological activities of such transcripts or translation products.

It is also an object of this invention to provide screening assays using Zis-SR which can identify compounds which have therapeutic benefit for the secretory pathway. This invention also claims those compounds, the use of these compounds in treating disorders or conditions associated with defects in the regulated secretory pathway, and any use of any compounds identified using such a screening assay in treating disorders or conditions associated with defects in the regulated secretory pathway.

Generally, high throughput screens Zis-SR modulators i.e. candidate or test compounds or agents (e.g., peptides, peptidomimetics, small molecules or other drugs) may be based on assays which measure biological activity of Zis-SR. The invention therefore provides a method (also referred to herein as a “screening assay”) for identifying modulators, which have a stimulatory or inhibitory effect on, for example, Zis-SR biological activity or expression, or which bind to or interact with Zis-SR proteins, or which have a stimulatory or inhibitory effect on, for example, the expression or activity of Zis-SR interacting proteins (targets) or substrates.

In one embodiment, the invention provides assays for screening candidate or test compounds which interact with substrates of a Zis-SR protein or biologically active portion thereof.

In another embodiment, the invention provides assays for screening candidate or test compounds which bind to or modulate the activity of a Zis-SR protein or polypeptide or biologically active portion thereof.

In one embodiment, an assay is a cell-based assay in which a cell which expresses a Zis-SR protein or biologically active portion thereof, either natural or recombinant in origin, is contacted with a test compound and the ability of the test compound to modulate Zis-SR biological activity, e.g., modulation of secretion, or any other measurable biological activity of Zis-SR is determined. Determining the ability of the test compound to modulate Zis-SR activity can be accomplished by monitoring, for example, CPE immunoreactivity the release of a hormone or other compound, formation of secretory granules, release of secretory granules upon cellular depolarization from a cell which expresses Zis-SR, homolog, fragment or variant thereof.

In another embodiment, the assay is a cell-based assay comprising a contacting of a cell containing a target molecule (e.g. another molecule, substrate or protein that interacts with or binds to Zis-SR) with a test compound and determining the ability of the test compound to indirectly modulate (e.g. stimulate or inhibit) the biological activity of Zis-SR by binding or interacting with the target molecule. Determining the ability of the test compound to indirectly modulate the activity of Zis-SR can be accomplished, for example, by determining the ability of the test compound to bind to or interact with the target molecule and thereby to indirectly modulate Zis-SR, to modulate secretory granule formation, or to modulate other biological activities of Zis-SR. Determining the ability of the Zis-SR protein or a biologically active fragment thereof, to bind to or interact with the target molecule can be accomplished by one of the methods described above or known in the art for determining direct binding.

In yet another embodiment, an assay of the present invention is a cell-free assay in which a Zis-SR protein or biologically active portion thereof, either naturally occurring or recombinant in origin, is contacted with a test compound and the ability of the test compound to bind to, or otherwise modulate the biological activity of, the Zis-SR protein or biologically active portion thereof is determined. Preferred biologically active portions of the Zis-R proteins to be used in assays of the present invention include the extended SR region of Zis-SR. Binding of the test compound to the Zis-SR protein can be determined either directly or indirectly as described above.

The cell-free assays of the present invention are amenable to use of both soluble and/or membrane-bound forms of isolated proteins. In the case of cell-free assays in which a membrane-bound form of an isolated protein is used (e.g. a sodium channel) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton®X-114, Thesit®), Isotridecypoly(ethylene glycol ether)n. 3-[(3-cholamidopropyl)dimethyamino]-1-propane sulfonate (CHAPS), 3-[(3-cholamidopropyl)-dimethylamino]-2-hydroxy-1-propane sulfonate (CHAPSO), or N-dodecyl-N,N-dimethyl-3-ammnonio-1-propane sulfonate.

In more than one embodiment of the above assay methods of the present invention, it may be desirable to immobilize either Zis-SR or a target molecule thereof to facilitate separation of complexed from uncomplexed forms of one or both of the proteins, as well as to accommodate automation of the assay. Binding of a test compound to a Zis-SR protein or interaction of a Zis-SR protein with a target molecule in the presence and absence of a candidate compound, can be accomplished in any vessel suitable for containing the reactants. Examples of such vessels include microtitre plates, test tubes and micro-centrifuge tubes. In one embodiment a fusion protein can be provided which adds a domain that allows one or both of the proteins to be bound to a matrix. For example, glutathione-S-transferase/Zis-SR fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or Zis-SR protein and the mixture incubated under conditions conducive to complex formation (e.g. at physiological conditions for salt and pH). Following incubation the beads or microtitre plate wells are washed to remove any unbound components, the matrix immobilized in the case of beads, complex determined either directly or indirectly, for example, as described above. Alternatively, the complexes can be dissociated from the matrix, and the level of Zis-SR binding or activity determined using standard techniques.

Other techniques for immobilizing proteins on matrices (and well-known in the art) can also be used in the screening assays of the invention. For example, either a Zis-SR protein or a Zis-SR target molecule can be immobilized utilizing conjugation of biotin and streptavidin. Biotinylated Zis-SR protein or target molecules can be prepared from biotin-NHS(N-hydroxysuccinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with Zis-SR protein or target molecules but which do not interfere with binding of the Zis-SR protein to its target molecule can be derivatized to the wells of the plate, and unbound target or Zis-SR protein trapped in the wells by antibody conjugation. Methods for detecting such complexes, in addition to those described above for the GST-immobilized complexes, include immunodetection of complexes using antibodies reactive with the Zis-SR protein or target molecule, as well as enzyme-linked assays which rely on detecting an enzymatic activity associated with the Zis-R protein or target molecule.

In another embodiment, modulators of Zis-SR expression are identified in a method wherein a cell is contacted with a candidate compound and the expression of Zis-SR mRNA or protein in the cell is determined. The level of expression of Zis-SR mRNA or protein in the presence of the candidate compound is compared to the level of expression of Zis-SR mRNA or protein in the absence of the candidate compound. The candidate compound can then be identified as a modulator of Zis-SR expression based on this comparison. For example, when expression of Zis-SR mRNA or protein is greater (statistically significantly greater) in the presence of the candidate compound than in its absence, the candidate compound is identified as a stimulator of Zis-SR mRNA or protein expression. Alternatively, when expression of Zis-SR mRNA or protein is less (statistically significantly less) in the presence of the candidate compound than in its absence, the candidate compound is identified as an inhibitor of Zis-SR mRNA or protein expression. The level of Zis-SR mRNA or protein expression in the cells can be determined by methods described herein or other methods known in the art for detecting Zis-SR mRNA or protein.

The assays described above may be used as initial or primary screens to detect promising lead compounds for further development. Often, lead compounds will be further assessed in additional, different screens. Therefore, this invention also includes secondary Zis-SR screens which may involve secretion assays utilizing mammalian cell lines expressing Zis-SR.

Tertiary screens may involve the study of the identified modulators in rat and mouse models for diseases or conditions associated with defects in the secretory pathway. Accordingly, it is within the scope of this invention to further use an agent identified as described herein in an appropriate animal model. For example, an test compound identified as described herein (e.g., a Zis-SR modulating agent, an antisense Zis-SR nucleic acid molecule, a Zis-SR-specific antibody, or a Zis-SR-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment, as described herein.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261 :1303; Carrell et al. (1994) Angew. Chem, Int. Ed Engl. 33:2059; Carell et al. (1994) Angew. Chem. Jnl. Ed. Engl. 33:2061; and in Gallop et al. (1994). Med Chem. 37:1233. Libraries of compounds may be presented in solution (e.g. Houghten (1992) Biotechniques 13:412421). or on beads (Lam (1991) Nature 354:82-84), chips (Fodor (1993) Nature 364:555-556). bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91: 11422; Zuckermann et al. (1994), J: Med. Chem. 37:2678; Cho et al. (1993), Science 261 :1303; Carrell et al. (1994) Angew. Chem Int. Ed. Engl. 33:2059, or luciferase, and the enzymatic label detected by determination of conversion of an appropriate substrate to product.

In summary, based on the disclosure herein, those skilled in the art can develop Zis-SR screening assays which are useful for identifying compounds which are useful for treating diseases or conditions associated with a defect in the regulated secretory pathway in cells. The assays of this invention may be developed for low-throughput, high-throughput, or ultra-high throughput screening formats.

The assays of this invention employ either natural or recombinant Zis-SR protein. Cell fraction or cell free screening assays for modulators of Zis-SR biological activity can use in situ, purified, or purified recombinant Zis-SR proteins. Cell based assays can employ cells which express Zis-SR protein naturally, or which contain recombinant Zis-SR gene constructs, which constructs may optionally include inducible promoter sequences. In all cases, the biological activity of Zis-SR can be directly or indirectly measured; thus modulators of Zis-SR biological activity can be identified. The modulators themselves may be further modified by standard combinatorial chemistry techniques to provide improved analogs of the originally identified compounds.

Finally, portions or fragments of the Zis-SR cDNA sequences identified herein (and the corresponding complete gene sequences), and in particular the extended SR domain of Zis-SR, can be used in numerous ways as polynucleotide reagents. For example, these sequences can be used to: (i) map their respective genes on a chromosome and thus, locate gene regions associated with genetic disease (mutations/polymorphisms) related to secretory disorders that involve Zis-SR directly or indirectly; (ii) identify an individual from a minute biological sample (tissue typing); and (iii) aid in forensic identification of a biological sample.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the effects of stimulation of the CAMP signaling pathway on 6T3 cells. Stimulation with 8-bromo-cAMP for 24 hours has an important effect on the formation of secretory granules , as shown in B, C and D. Unstimulated 6T3 cells shown in A, do not display any secretory granules;

FIG. 2 shows the representative DD-PCR of stimulated and unstimulated 6T3 cells, showing upregulation of cDNA fragments obtained. The bands are excised and re-amplified. The cDNAs obtained are used to screen Northern blots and to carry out in situ hybridization analysis. The cDNA fragment is sequenced and a full length clone can be obtained using standard RACE PCR methodologies;

FIG. 3 shows the Northern blot analysis of Zis-SR showing its upregulation in 6T3 cells after stimulation with CAMP. Lane A is RNA extracted from unstimulated 6T3 cells, showing basal levels of Zis-SR. Lane B shows RNA from stimulated 6T3 cells with strong upregulation of Zis-SR mRNA;

FIG. 4 shows the nucleotide sequence of Zis-SR. A total of 2616 nucleotides of Zis-SR were sequenced. The open reading frame of 330 amino acids is shown. Start and stop codons are shown in bold. The dashed line indicates the position of the two zinc finger motifs. The boxed amino acids represent the nuclear localization signal. The underlined region is the SR domain. The bold underlined sequence is the polyadenylation site;

FIG. 5 shows the comparative amino acids analysis of mouse Zis-SR (in bold) with mouse and human Zfp265, human Zis1 and Zis2, rat Zis, and Xenopus C4SR. The C-terminal region of mouse Zis-SR contains a unique extended SR domain. The closest homology is found in the Xenopus C4SR C-terminal region, which is also shown in bold. Identical amino acids between mouse Zs-SR and the Xenopus C4SR C-terminal region are boxed. The “.” indicates an identical amino acid between all the aligned sequences. Dashed lines indicate gaps;

FIG. 6 shows an alignment of mouse Zis-SR, and its closest homologous as found in the databases, including the Xenopus C4SR and the C. Elegans gene Y25C1A.8. In each case, a C-terminal extended SR domain is observed.

FIG. 7 shows the PCR and sequence analysis of frame shift observed between Zis and Zis-SR. The three sequencing gels are of PCR-amplified regions of Zis-SR in three separate species, human rat and mouse. The three cytosine nucleotides are observed in all cases. Below is the comparative alignment showing the frame shift cause by the additional cytosine nucleotide, when comparing the reported rat Zis sequence and Zis-SR. This frame shift is responsible for the extended SR domain in Zis-SR;

FIG. 8 shows the alignment of the zinc finger motifs of Zis-SR with similar motifs in C4SR and other proteins containing zinc finger motifs. Athal-1 and Athal-2: zinc fingers from an Arabidopsis thaliana putative protein (Genbank AC003952). C4SR-1 and C4SR-2: zinc fingers from Xenopus laevis RNA-binding protein (Genbank X96469). mZis-1 and mZis-2: zinc fingers from mouse Zis-SR cDNA clone. Spombehyp-1 and Spombehyp-2: zinc fingers from Saccharomyces pombe putative protein (Genbank Z98597). ARP-1 and ARP-2: zinc fingers from Saccharomyces cerevisiae hypothetical protein (Genbank Z67750). EWS: zinc finger from human EWS oncoprotein (Genbank X79233);

FIG. 9 shows the expression of Zis-SR in various endocrine and non-endocrine cell lines and tissues as determine by Northern blot analysis. Zis-SR is highly expressed in brain tissues. Expression is also observed in the pancreas and pancreas-derived cell lines (i.e., RINm5F and βTC3 cells);

FIG. 10 shows the distribution of Zis-SR in the mouse CNS as determined by in situ hybridization histochemistry. An ³⁵S-labeled cRNA probe was used for detection of Zis-SR mRNA. (A) Zis-SR mRNA was most highly expressed in cortex (Cx), hippocampus, anterior cortical amygdoloid nucleus (Aco) and ventral posterior thalamic nucleus (VP). (B) the expression of Zis-SR mRNA was observed in posteromedial cortical amygdoloid nucleus (PMCo), substantia nigra compact (SNC), geniculate body (GB) as well as scattered hilar cells (Hil);

FIG. 11 shows the expression of Zis-SR in the mouse pituitary and brain. In situ hybridization with an ³⁵S-labeled cRNA probe demonstrated that relatively high levels of Zis-R mRNA were expressed in: (A) the pituitary intermediate lobe (IL) and (B) field CA3 of ammon's horn (CA3). (C) Bright field image showing neuronal expression of Zis-SR in the cortex. Open arrows: glial cells, plain arrows: neurons (D) Control hybridization with sense probe;

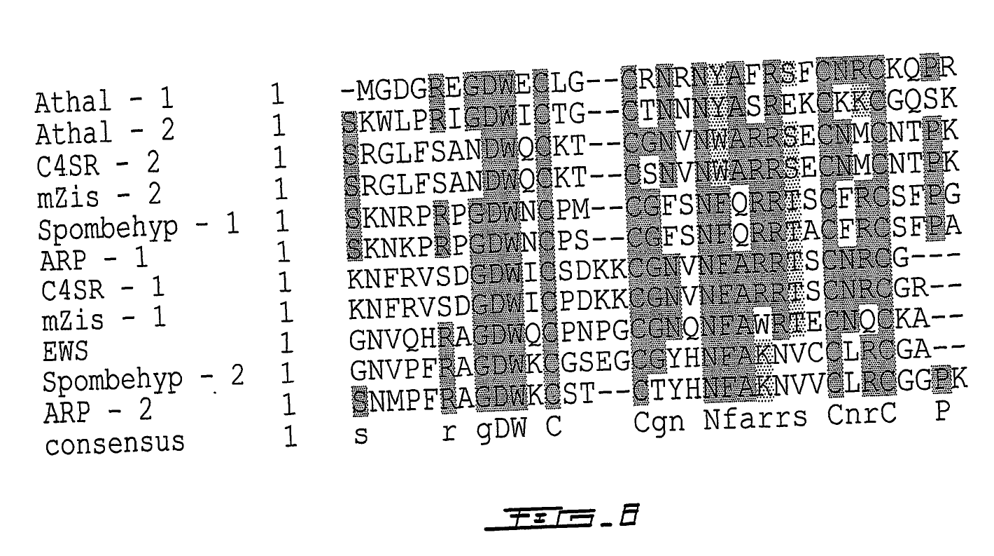

FIG. 12 shows the analysis of Zis-SR mRNA expression in stable AtT-20 cells lines expressing different antisense constructs of Zis-SR, including 400, 700, 1400 and full length (FL) nucleotides. All constructs were expressed using pcDNA3.1-hygro (Invitrogen). Various clones are observed to have significant reduction in Zis-SR levels as compared to control AtT-20 cells. The best clones were clones 700 As-#3 and 1400 As #1, expressing only 20-22% of normal Zis-R mRNA levels;

FIG. 13 shows the CPE immunoreactivity in (A) and (B) AtT-20 control cell line and (C) and (D) AtT-20 As cell line. Arrows in (A) and (B) show examples of CPE staining at the tips of AtT-20 extensions, while in (C) and (D) no staining is observed. CPE perinuclear (TGN) staining can still be observed in both control AtT-20 and AtT-20-As cells;

FIG. 14 shows (A) Northern and (B) Western blot analysis of selected antisense (As) stable cell lines for CPE mRNA expression and CPE protein content. The Northern blot analysis reveals that no change in CPE mRNA is observed in cells expressing As-Zis-SR. However, CPE protein content is significantly reduced, especially in cell lines where Zis-SR is most affected. This data reflects the fact that although CPE expression is unaffected, its content is reduced due to lower storage capacity of the Zis-As stable cell lines (i.e., 700 As-3);

FIG. 15 shows the Western blot analysis of CPE immunoreactivity in the media of AtT-20 cells and the stable cell line 700 As-3 (i.e., expressing the Zis-SR antisense construct). The cells were depolarized using 56 mM KCl for 30 min. The experiment is carried out in triplicate. In the top panel, increased CPE immunoreactivity is observed in the media after depolarization. In the bottom panel, basal release (5 mM KCl) of CPE immunoreactivity is higher and is not affected by depolarization. This data demonstrates that the regulated secretory pathway has been disrupted in the 700 As-3 AtT-20 cell line, due to the reduced expression of Zis-SR; and

FIG. 16 shows the ultrastructural analysis of control (A) AtT-20 cells and (B) AtT-20-As cells. Control AtT-20 cells (on the left) show typical secretory granules (see arrows), while AtT-20-As cells (on the right), which have much lower levels of Zis-SR, show no evidence of secretory granules.

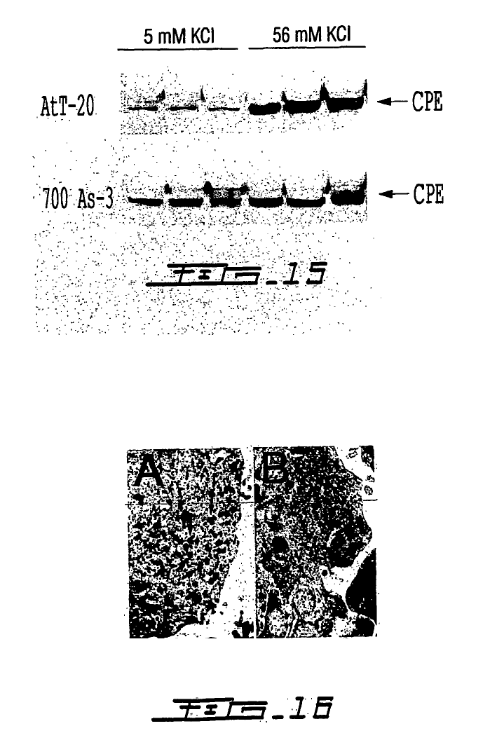

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

To identify the genes involved in the differentiation of the neuroendocrine phenotype, a cell line named HYA.15.6.T.3 (6T3) was used. The 6T3 cell was derived from the fusion of a mouse myeloma cell line (4T001) and the anterior pituitary corticotroph cell line (AtT-20). Previously the 6T3 cell was used to study trafficking of cell membranes and was not considered as a neuroendocrine cell line due to its lack of secretory granules. Thus the 6T3 cell line did not have the most important component of the neuroendocrine phenotype.

Upon appropriate stimulation and correct culture conditions, the 6T3 cells can be made to differentiate into a neuroendocrine phenotype. There is evidence to show that under basal conditions these cells do not produce or store secretory granules and thus do not display the neuroendocrine phenotype. But upon stimulation of these cells with specific neuroendocrine differentiating agents, the cells show all the characteristics of the neuroendocrine phenotype including the presence of secretory granules.

In accordance with the method of the present invention, a gene involved in the cellular differentiation leading to the neuroendocrine phenotype was identified. The method involves exposing the cells to cAMP and then determining all differentially expressed genes as compared to cells that were not exposed to cAMP. The genes that are isolated are expressed in non-stimulated 6T3 cells to show that they carrying out neuroendocrine differentiation. The isolated genes are also used to block cellular differentiation using anti-sense approaches. The method also permits the identification of genes that modulate the formation of the regulated secretory pathway. The identified genes would then be used to transform embryonic stem cells that would be used for transplantation and replacement cell therapy. As a proof of the concept, a gene that codes for a previously uncharacterized mammalian protein which was named Zis-SR was cloned. Zis-SR is a homolog of the Zis gene, which is involved in RNA splicing. We show that Zis-R is vital to the formation of secretory granules.

Stimulation of 6T3 Cells with cAMP Results in the Formation of Functional Secretory Granules

The 6T3 cell line originates from fusion experiments using the mouse pituitary cell line AtT-20 and the mouse myeloma 4T001 (Matsuuchi et al., 1991). AtT-20 cells are extremely well characterized and known to express a number endocrine-related genes, including proopiomelanocortin (POMC), pro-protein convertases, carboxypeptidase E (CPE), peptidyl amidating monooxygenase (PAM), chromogranins (i.e., CgA, CgB, Sgll), and the neuroendocrine polypeptide 7B2. Each of these molecules is directed to the regulated secretory pathway of endocrine cells. AtT-20 cells also package these proteins in electron dense secretory granules. However, the first study describing 6T3 cells (Matsuuchi et al., 1991) revealed that they did not express various soluble markers of the dense core vesicles nor could dense core secretory granules be identified therein. It was therefore of interest to characterize the 6T3 cell line as to expression levels of various endocrine-related proteins (i.e., processing enzymes, chromogranins and hormones) that are found in the regulated secretory pathway of the parental AtT-20 cells (Day et al., 1995). It was determined that 6T3 cells were completely devoid of POMC. The 6T3 cells also had much lower levels of CgA, CgB and Sgll than AtT-20 cells. The levels of processing enzymes such as CPE, and furin were unchanged, while PC1/PC3 levels were reduced. Based on these results and others, it was concluded that 6T3 cells retain some characteristics of endocrine cells (i.e., since some endocrine markers are still present at various levels). However, the lack of secretory granules and regulated secretory function suggested the presence of specific defects affecting compartmentalization but not necessarily the expression of all endocrine markers. If these defects could be identified, it could lead us to an understanding of important cellular processes related to the biogenesis and functionality of secretory granules.

Experiments to investigate the capacity of 6T3 cells to regulate expression of endocrine markers in comparison to the parental AtT-20 cells were also conducted. 6T3 cells were stimulated with cAMP for various periods of time. Morphological changes were noted following cAMP stimulation. Similar to AtT-20 cells, stimulated 6T3 cells demonstrated the ability to form extensions and processes. It was also noted that POMC mRNA expression was restored in stimulated 6T3 cells (Day et al., 1995). This surprising but important clue suggested that defects associated with the 6T3 cell (i.e., such as the silencing of POMC expression) could be restored with cAMP stimulation. If this was true, then it seemed possible that other defects could also be corrected. It thus seemed possible that cAMP stimulation could restore a functional regulated secretory pathway in 6T3 cells.

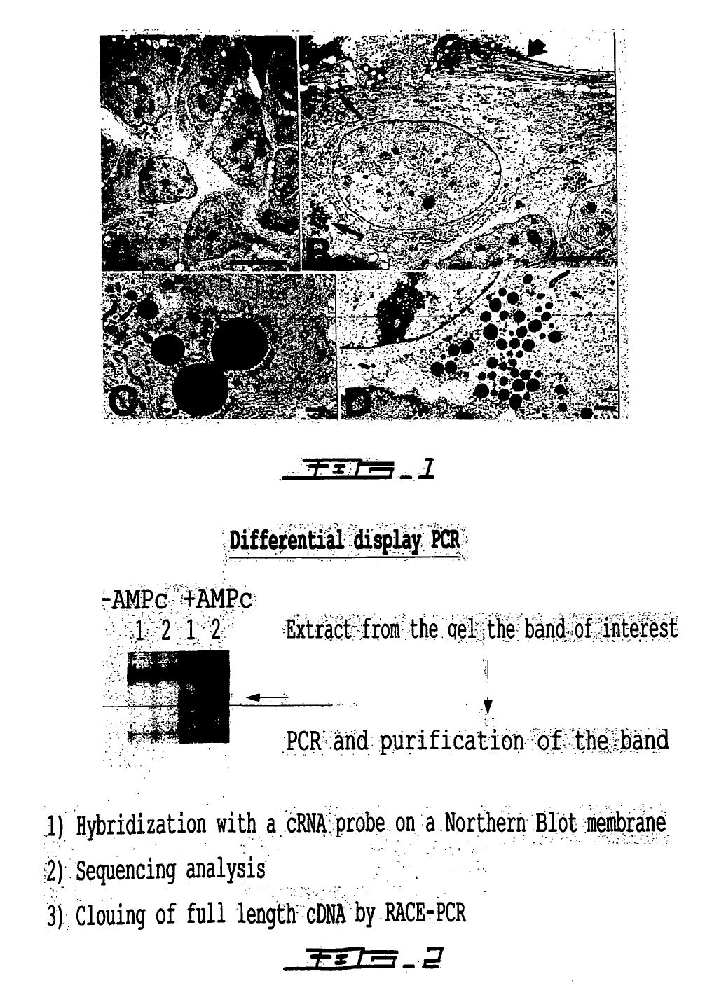

To investigate this possibility, cAMP-stimulated 6T3 cells were examined at the ultrastructural level for any evidence of secretory granules. This analysis revealed the presence of dense-core granules in the cytoplasm and the extremities of treated 6T3 cells (FIG. 1). If these newly formed dense core secretory granules were indeed genuine structures, a detection of the storage of hormones within their dense cores could be carried-out. An immunohistochemical analysis of cAMP-stimulated 6T3 cells at the ultrastructural level was thus performed. As mentioned above, non-stimulated 6T3 cells do not express POMC, but after cAMP treatment, POMC expression is induced in 6T3 cells. The distribution of immunoreactive β-LPH, a cleavage product of POMC, in cAMP-stimulated 6T3 cells was therefore examined. This analysis showed β-LPH immunoreactivity within newly formed secretory granules, thus demonstrating that they were authentic storage compartments that had been induced by CAMP stimulation.

Since it could be shown that cAMP-stimulated 6T3 cells could re-form dense core secretory granules and correctly cleave and store newly induced POMC protein, it was important to verify if regulated secretion was now functional. In other words, once these dense core secretory granules were formed, did they have the capacity to fuse with the external membrane and release their contents into the media upon stimulation. These experiments were carried out on 6T3 cells that had been induced with cAMP to produce POMC and dense core secretory granules. After a washout period (i.e., 24 hrs to remove all secretory effects of cAMP stimulation), the induced 6T3 cells were treated with a short-term depolarization stimulus (i.e., 15 min of 56 mM KCl). The media was examined for the release of P-endorphin immunoreactivity. Such a short term stimulus can only release stored products of the cell and will not be sufficient to influence transcriptional changes which could blur the sought after effect. The data showed a rapid release of p-endorphin immunoreactivity in the media as compared to controls. These experiments thus demonstrated that cAMP differentiated 6T3 cells could release their newly formed dense core secretory granules through a normal regulated secretory mechanism involving exocytosis. These data also suggested that the secretion-defect(s) associated with 6T3 cells were related to the formation of secretory granules and not to a defective exocytotic machinery. It therefore seemed possible that the 6T3 secretion defects lied upstream, preventing the formation of secretory granule formation.

Thus 6T3 cells have specific defects in terms of full neuroendocrine differentiation, which can be repaired after specific stimulation with agents that affect the cAMP-related signaling pathways. These agents include 8-bromo-cAMP, dibutryl cAMP and ligands which interact with seven transmembrane receptors that activate G proteins and the adenyate cyclase, as for example croticotrophin releasing hormone (CRH). Stimulation by any of these agents results in the differentiation of 6T3 cells as determined by the appearance of distinct secretory granules as observed in ultrastructural studies. Furthermore, these newly formed granule storage compartments can be shown to contain authentic peptides and proteins that are normally directed to secretory granules, such as β-lipotrophin (β-LPH) or β-endorphin. Finally, these secretory granule components are released from the 6T3 cells upon cellular depolarization induced with short-term treatment with KCl. This demonstrates that the newly formed secretory granules are fully functional in 6T3 cells.

Identification of 6T3 Defects Using Differential Display Polymerase Chain Reaction (DD-PCR).

The fact that stimulating 6T3 cells with cAMP results in the formation of functional secretory granules provides the means to carry-out plus/minus screening. Indeed, it now became possible to try to define the molecular factors that are playing a role in the induction of regulated secretion by assessing the difference between non-stimulated and stimulated 6T3 cells. Current methods to distinguish mRNAs in comparative studies rely largely on differential or subtractive hybridization techniques. Several important genes implicated in tumorigenesis have been isolated using these method. Although subtraction is quite sensitive and can detect fairly rare mRNAs, the method recovers genes incompletely and selects for genes in only one direction at a time during a two-way comparison between a pair of cells. This process can be laborious as well as time-consuming. DD-PCR (GenHunter) was developed with the goal of identifying differentially expressed genes or detecting individual mRNA species that are changed in mammalian cells, then recovering and cloning the cDNA. This method utilizes PCR amplification and denaturing polyacrylamide gel electrophoresis, two of the most commonly used molecular biological methods, and provides a sensitive and straightforward approach to detect differentially expressed genes. DD-PCR (Liang et al., 1998) was thus chosen with the aim of identifying genes involved in the formation of secretory granules.

6T3 cells were stimulated with 5 mM cAMP and non-stimulated 6T3 cells were used as control. Total RNA was extracted from treated and untreated cells. The RNA from each sample was reverse transcribed to prepare cDNA from each sample. The cDNA was then amplified by PCR with a set of anchored oligo (dt) primers and an arbitrary decamer. Specifically, an RNA sample is reverse transcribed with each of 4 sets of degenerate anchored oligo (dt) primers (T12MN), where M can be G, A, or C and N is G, A, T and C. Each primer set is dictated by the 3′ base (N), with degeneracy in the penultimate (M) position. For example, the primer set where N=G consists of:

5′-TTTTTTTTTTTTGG-3′
5′-TTTTTTTTTTTTAG-3′
5′-TTTTTTTTTTTTCG-3′

The resulting cDNA population was PCR amplified using the degenerate set, an arbitrary decamer and radioactive nucleotide. The radioactively labeled PCR products that represent a subpopulation of mRNAs defined by a given primer set are separated on a denaturing polyacrylamide gel. By changing primer combinations, most of the RNA species in a cell may be represented. Side by side comparisons of RNA samples from treated and untreated samples allows the identification and cloning of differentially expressed genes.

This method is extremely rapid and false positives can be rapidly screened out by a simple test on Northern blot. In other words once a band is observed as being differentially displayed, that band can be isolated, re-amplified with the same primers and a cDNA probe can be made and tested on Northern blots that were prepared from the original RNA samples. Once a band is confirmed and shown to be regulated on the Northern blot, it can be sequenced and compared to the existing databases for homologies. If it is a known gene then it can be used to clone in further studies (e.g. expression in 6T3 cells, precise localization in the pancreas or other tissues by in situ hybridization, etc.). If it is not found in the existing databases, its tissue localization in endocrine tissues (e.g., pancreas, pituitary, adrenal) and in endocrine and non-endocrine cell lines that are available can be examined (Seidah et al., 1994). If the gene is of interest, a full-length clone can be obtained in order to express the protein and further define its function.

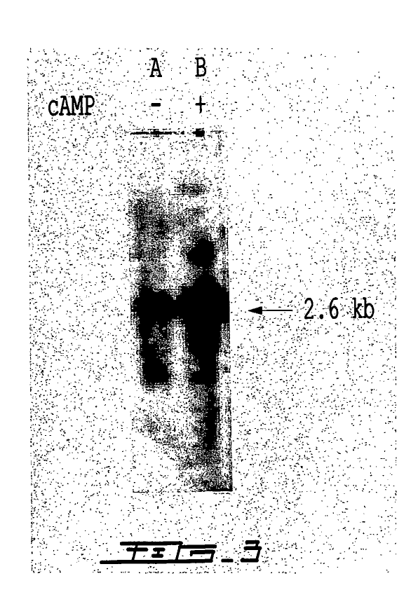

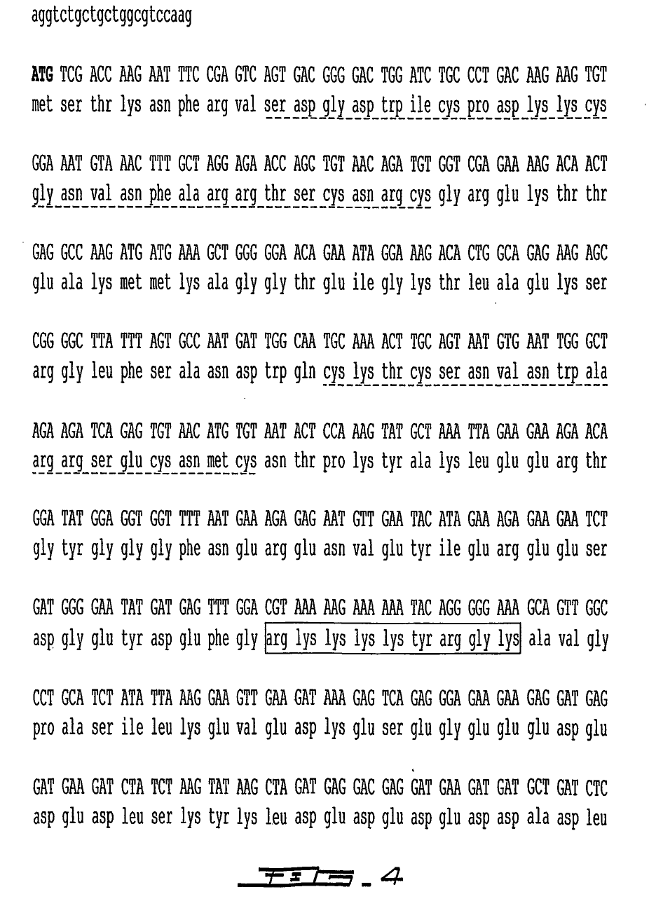

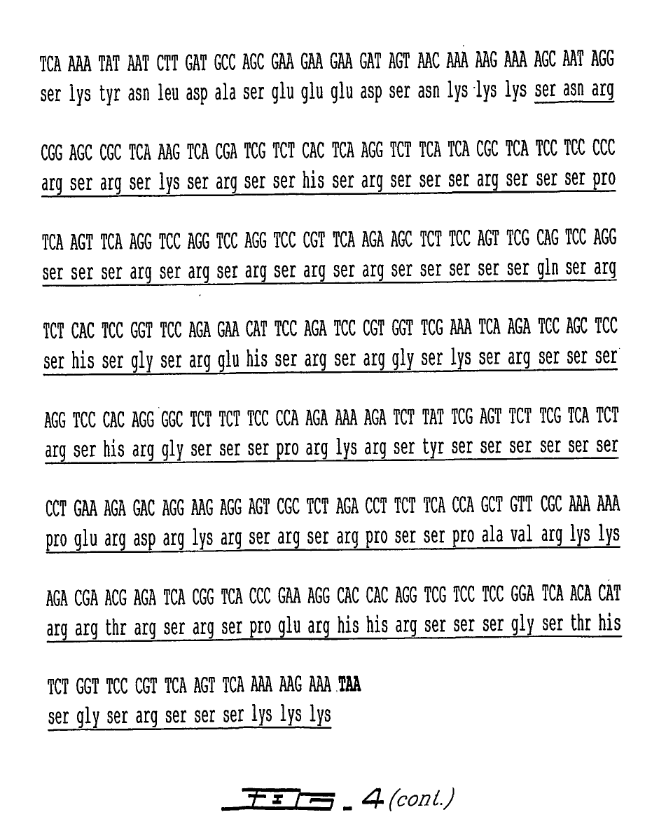

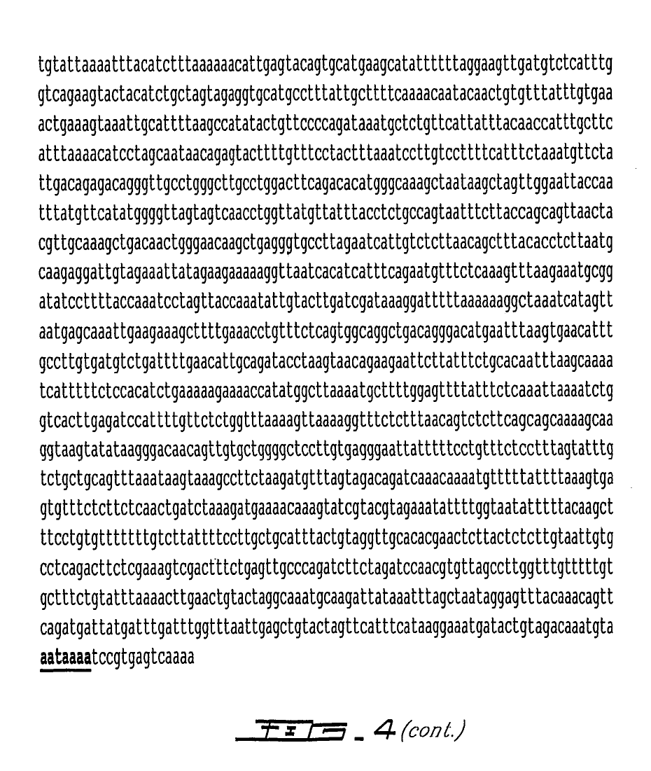

A differential display-PCR methodology (Liang et al., 1998) to compare stimulated and non-stimulated 6T3 cells was thus used (FIG. 2). Various cDNA fragments were isolated based on the increased intensity of the observed amplified bands. That these cDNAs corresponded to up-regulated genes by further Northern blot analysis screening was confirmed (FIG. 3). Although a number of candidate genes from this first round of screening experiments were retained, the instant invention focuses on one gene having particular interest. This mouse gene was named Zis-SR (zinc finger splicing with extended Ser-Arg domain) to take into account its strong homology with the reported Zis gene (Karginova et al., 1997). Of note, Zis-SR differs from Zis by virtue of an extended SR domain (FIG. 4 and FIG. 5), of 68 amino acids starting at amino acid position 243 (His) in mZis-SR.

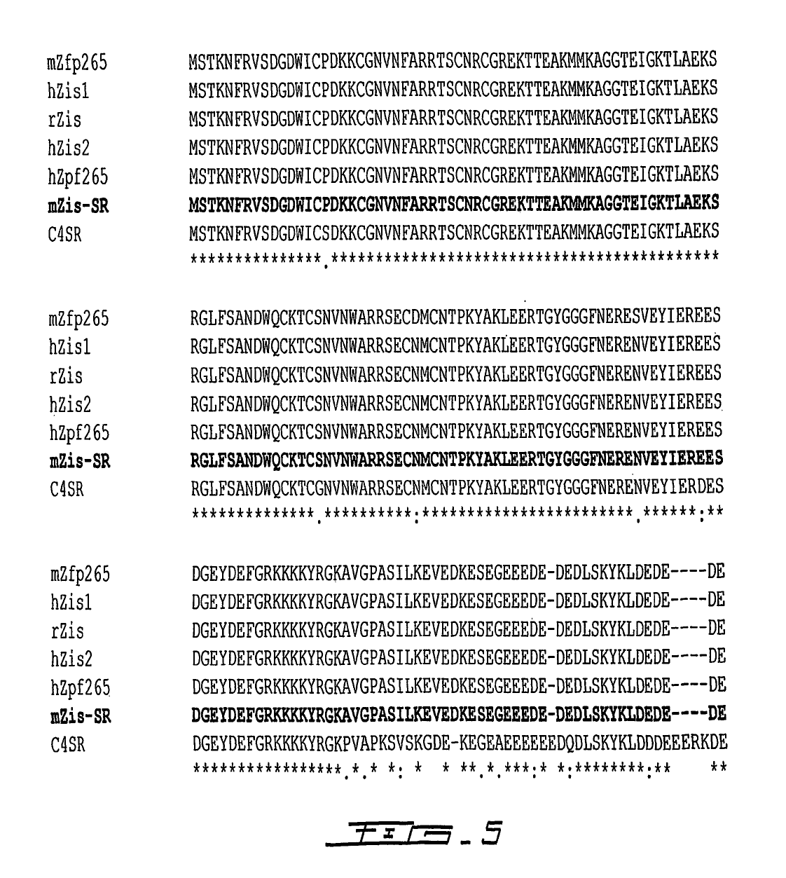

Using the nucleotide sequence information obtained from the partial DD-PCR fragment, oligo primers were synthesized and were used to obtain the full-length cDNA of Zis-SR. RACE-PCR cloning was carried out on Marathon-Ready cDNAs (Clontech Laboratories). The full length Zis-SR cDNA was 2,616 nts which corresponds to the size observed by Northern blot analysis. A search of the gene databases revealed that Zis-SR is a homolog of Zis (Karginova et al., 1997). At the nucleotide level Zis-SR and Zis are virtually identical, with important differences that result in the translation of very distinct C-terminal domains (FIG. 5). Indeed, Zis-SR contains an extended SR domain that terminates at the stop codon, while Zis has a much shorter SR domain which does not extend into the C-terminal. In essence therefore, it seemed that the sequences of Zis shifted the reading frame of the protein such that it was significantly truncated at the C-terminus. Indeed, a comparison of the sequences of Zis and Zis-SR at the nucleotide level shows the presence of three cytosine nucleotides in the Zis-SR as compared to two cytosine nucleotides reported in the Zis sequence at positions 802-803 (numbering based on Zis rat sequence; Genbank Accession number AF013967). In order to verify the sequence data, a specific PCR test was designed so as to distinguish between Zis-SR and Zis and to confirm that Zis-SR is expressed in mouse, rat and human brain tissue (FIG. 6). Taken together, the results presented herein enable the conclusion that Zis-SR is a unique protein that had not been described previously in mammalian species. A search in non-mammalian species indicates a high homology of Zis-SR with the Xenopus C4SR both at the nucleotide and protein level. The function of C4SR is undetermined although its relationship to RNA splicing has been suggested (Ladomery et al., 2000). In a recent database search, homologs of Zis-SR in Drosophila and in Caenorhabditis elgans (C. elegans) were identified. Of note, in both species, the fully extended SR domain is conserved.

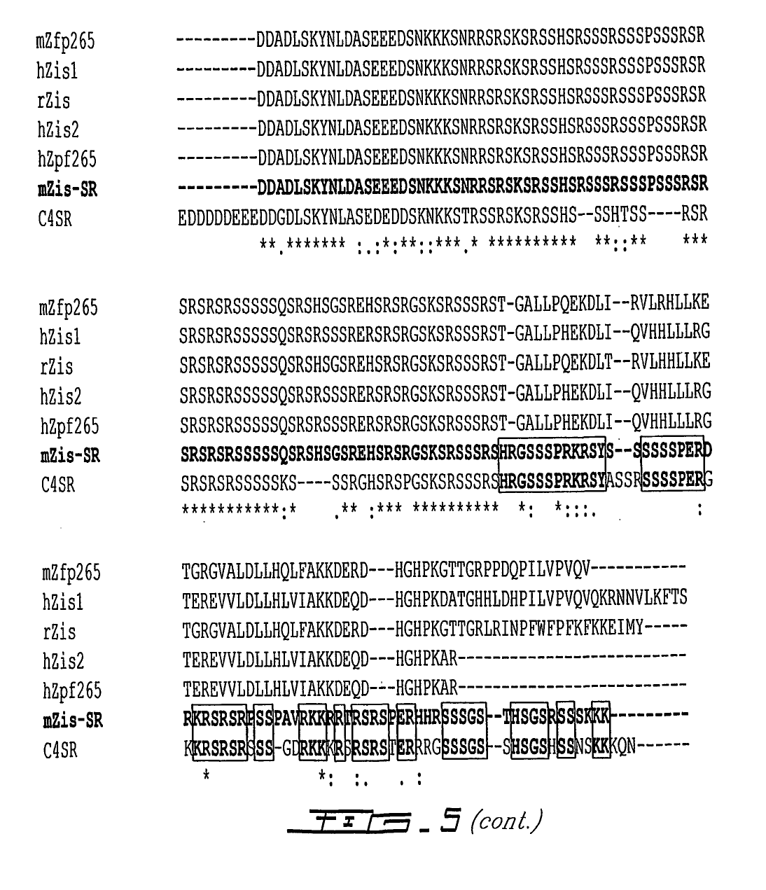

Zis-SR has an open reading frame coding for 330 amino acid protein with the following features: 1) an N-terminal tandem of zinc finger motifs, 2) a nuclear localization signal in the mid-portion of the protein and 3) a C-terminal domain rich in serine and arginine (SR domain). The SR domain is characteristic of nuclear RNA-binding proteins involved in the splicing of pre-mRNA (Fu et al., 1995; Manley et al., 1996). The SR domain of Zis-SR is twice the length of the SR domain of the previously described Zis protein. The analysis of the protein structure also shows the presence of two zinc finger motifs at the N-terminal portion of Zis-SR, one of which appears to contain a novel consensus motif (FIG. 7). Zinc fingers are classically found in transcription factors and typical serve to bind DNA. The function of these zinc finger domains is not known but could indicate the capacity of Zis-SR to bind DNA. This dual feature of zinc fingers and SR-domain makes Zis-SR a unique protein, unlike any other pre-mRNA splicing factor described to date. Unlike other RNA splicing factors, Zis-SR does not contain an RNA recognition motif, that permits the association of the splicing factor with RNA. It is also possible that the two zinc finger domains may be involved in RNA binding (Arranz et al., 1997; Caricasole et al., 1996; Clemens et al. 1993; Finerty et al., 1997; Shi et al., 1995). The exact function of Zis-SR remains to be formally tested experimentally. Without being bound by a particular theory, the most likely argument to date relating Zis SR to an RNA splicing function is its close homology to the Xenopus protein known as C4SR (Ladomery et al., 2000). The C4SR protein was isolated based on its RNA-binding properties (Karginova et al., 1997). Thus, the zinc fingers could be involved in RNA binding, while the extended SR domain of Zis-SR could be involved in the promotion of the spliceosome assembly by facilitating specific protein interactions and thus preventing random binding to RNA. Regulation of these specific molecular events could be dependent on the state of phosphorylation of Zis-SR (Yeakley et al., 1999).

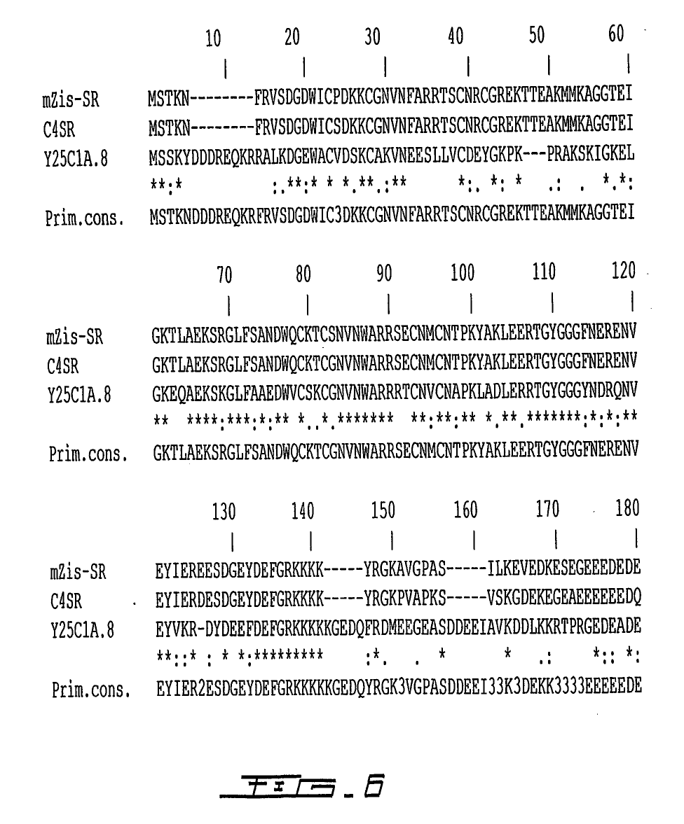

The analysis of the protein structure shows that the two zinc finger motifs of Zis/GAP1-5a are characteristic of a novel family of zinc finger proteins spread from yeast to mammals (FIG. 8). The consensus sequence of these Cys₂/Cys₂ zinc fingers is D-W—X—C—X₄⁻C—XₙC—N—X₆C—X₂—C. Interestingly, this zinc finger motifs occurs in the EWS (Crozat et al., 1993) and FUS oncoproteins, two human RNA-binding molecules. Unfortunately, the role of these zinc finger structures in EWS and FUS have not been studied yet in the context of RNA binding since EWS and FUS also bear other more classical RNA recognition motifs. This novel zinc finger structure which has been discovered has a defined biological role. The C4SR protein, the Xenopus homologue of Zis-SR, was isolated from its RNA-binding properties. Taken together, these observations suggest that both the SR domain and the zinc fingers of Zis-SR are related to RNA binding purposes.

Pre-mRNA splicing is a fundamental mechanism in all eukaryotic cells. Constitutive splicing is required for efficient protein expression by the removal of introns to form coherent coding sequence. Furthermore, alternative splicing of pre-mRNA is a powerful mechanism for the regulation of gene expression in eucaryotic cells that plays an important role in tissue development and cell differentiation. In Drosophila, a differentiation event as fundamental as sex determination is triggered by an alternative splicing mechanism mediated by the family of Transformer (TRA) alternative splicing factors (Tian et al., 1993). Genes can also be alternatively spliced to generate endocrine-specific isoforms. As an example, the isoform A of the subtilisin-like pro-protein convertase 5 (PC5) behaves like an endocrine protein, being sorted to the regulated secretory pathway, while the isoform B goes to the constitutive vesicular traffic (De Bie et al., 1996). It appears that a number of genes need an endocrine-specific pattern of splicing, requiring the presence of endocrine splicing factors. Since most of the known splicing factors are SR proteins, the discovery of an endocrine-specific protein with an SR domain like Zis-SR opens new insights for the study of endocrine differentiation.

SR proteins constitute a large family of nuclear phosphoproteins required for constitutive and regulated pre-mRNA splicing (Mayeda et al., 1992). The most studied members of this family are the constitutive splicing factors U1 snRNP 70 k and U2AF, as well as the positive regulators of alternative splicing SRp75, SRp55, SRp40, ASF/SF2, SC35, RBP1 and 9G8 (Chabot 1996). All of them comprise a domain rich in serines and arginines (SR). They also bear a RNA-recognition motif (RRM) which consists of a four-stranded antiparallel β-pleated sheet and two alpha helices packed on one side of the β sheet (Xu et al., 1997). Although some proteins with only the SR domains (no RRM's) have been shown to play a role in splicing mechanisms (Fetzer et al., 1997), only those with RRM's are called SR proteins.

Biochemical analysis of nuclear extracts suggests that a large number of different SR proteins are involved in splicing mechanisms (Blencowe et al., 1995; Neugebaur et al., 1995). Variations in the levels of different SR proteins often lead to a change in alternative mRNA splicing patterns (Gallego et al., 1997; Zahler et al., 1993). SR proteins have been shown to bind to purine-rich motifs in exons in pre-mRNA complexes, where they participate in the spliceosome assembly (Achsel et al., 1996). Their effect on pre-mRNA splicing is antagonised by type A/B hnRNP, another class of mRNA splicing factors (Hanamura et al., 1998).

SR proteins display a dynamic intranuclear organization that is regulated by phosphorylation of the serine residues in the RS domain. Several SR-protein kinases have been identified from diverse species (Duncan et al., 1997; Duncan et al., 1998; Gross et al., 1997; Gui et al., 1994b; Nayler et al., 1997). There is a fight linkage between the transcription process and the intranuclear organization of the splicing machinery (Bauren et al, 1996). Some SR proteins have been shown to directly interact with the highly phosphorylated C-terminal domain of RNA polymerase 11, being associated with the elongating RNA polymerase (Du et al., 1997; Kim et al., 1997), from where they translocate to the nascent transcripts to ensure efficient splicing, concomitant with transcription (Bourquin et al., 1997; Corden et al., 1997). The diversity of SR proteins is further enhanced by alternative splicing mechanisms, which can give several isoforms (Du et al., 1997). Some SR proteins have been shown to even regulate the splicing of their own mRNA (Jumaa et al., 1997; Sarkissian et al., 1996). The role of the SR domain is not clearly understood. While it is clear that the RRM is involved in RNA binding, the SR domain seems to play several roles. These include specificity of target RNA recognition (Misteli et al., 1998), mediation of the protein-protein interactions into spliceosome assembly (Tronchere et al., 1997; Wu et al., 1993), and nuclear sublocalisation of the splicing components (Caceres et al., 1997). In all cases, the degree of phosphorylation of the SR-domain has been shown to be critical for regulating function (Gui et al., 1994a; Kanopka et al., 1998).

Distribution of Zis-SR in Cell Lines and Tissues

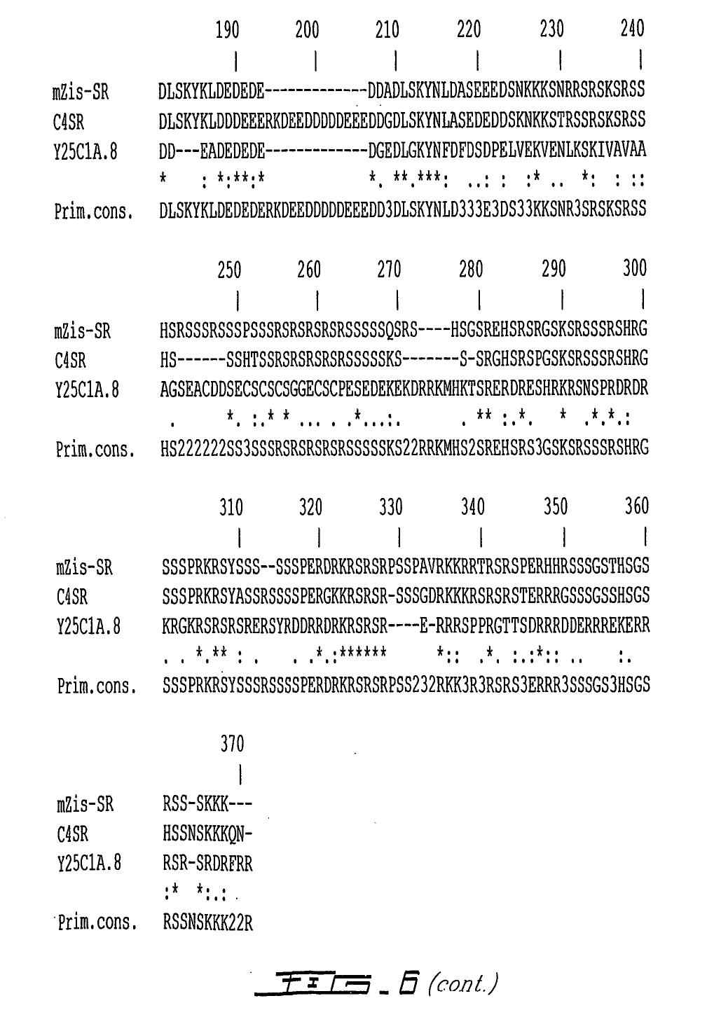

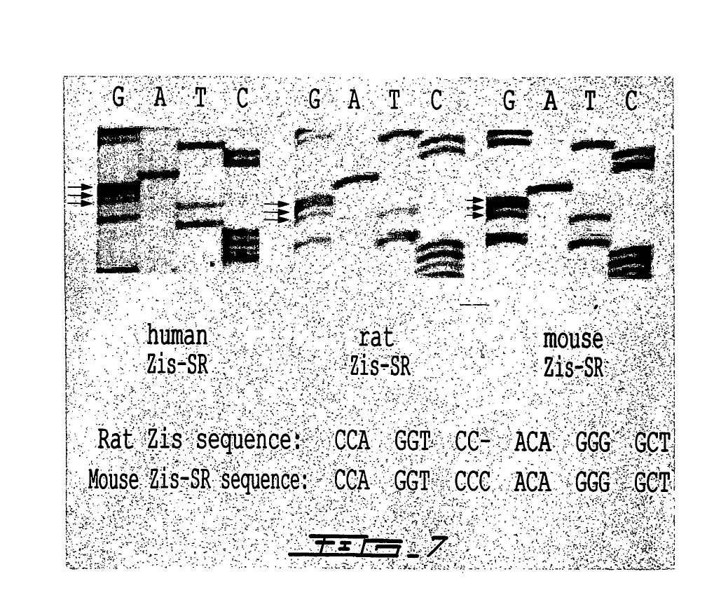

The distribution of Zis-SR mRNA in the mouse brain was examined by in situ hybridization. Zis-SR was exclusively expressed in neurons and not in glial cells (FIGS. 9 and 10). Furthermore, Zis-SR was preferentially expressed in all endocrine tissues investigated in the mouse (FIG. 9). Within stable cell lines, Zis-SR mRNA also had a distinct endocrine pattern (FIG. 9). This overall preferential expression pattern within neuro- and endocrine cell types is particularly interesting.

Direct Evidence that Zis-SR is Involved in Secretory Granule Biogenesis

Based on the strong correlation of Zis-SR expression with endocrine cells and tissues, an experiment was designed in order to directly address whether Zis-SR was implicated in the biogenesis of secretory granules, using gene silencing methodology in endocrine cell lines. AtT-20 cells are typical endocrine cells, that endogenously express Zis-SR and contain easily detectable secretory granules. AtT-20 cell lines expressing antisense Zis SR were thus created.

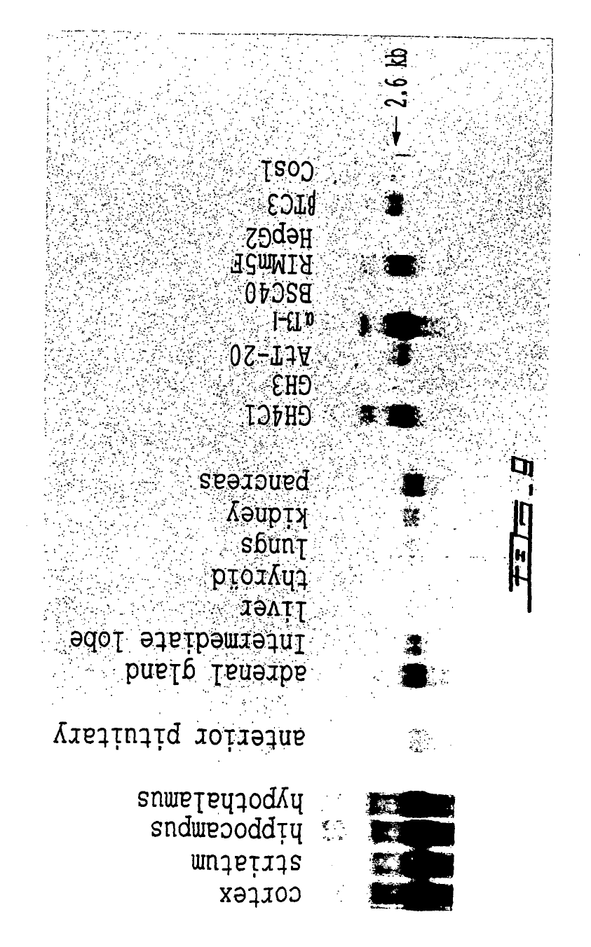

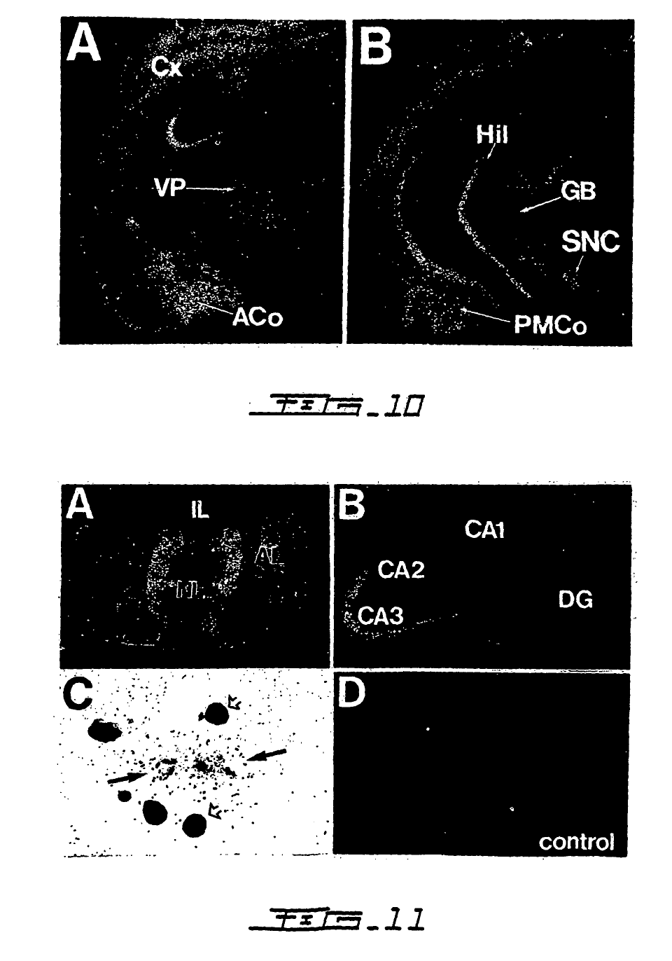

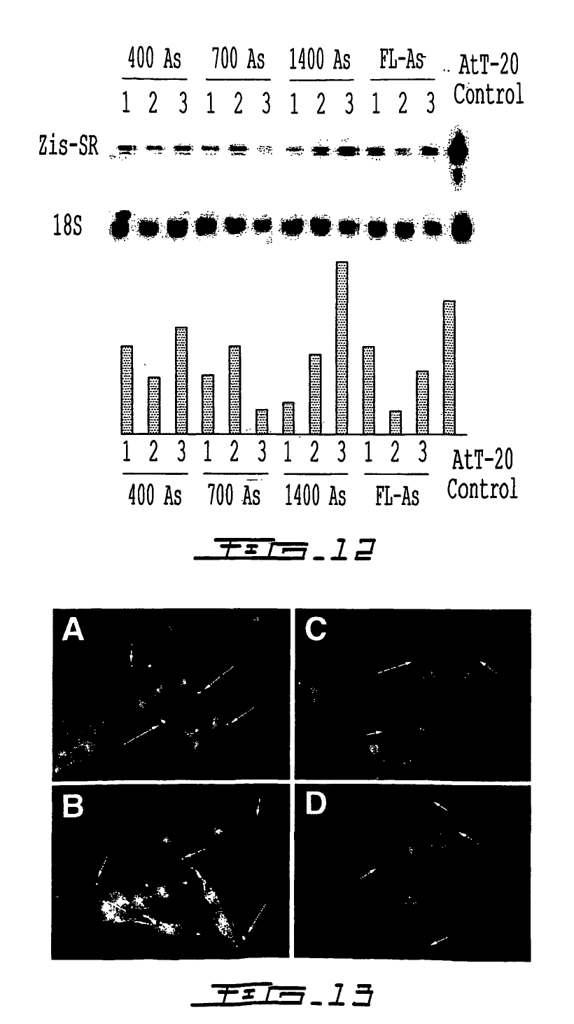

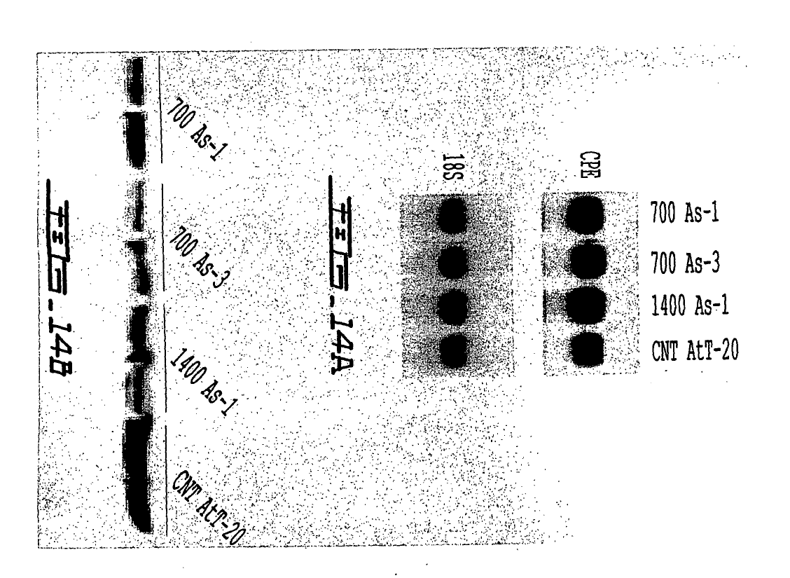

Stable AtT-20 cell lines expressing antisense cDNAs of Zis-SR were prepared. Different size fragments were tested including 400, 700 and 1400 nt long cDNAs, all inserted into the vector pcDNA3.1/Hygro. Various clones were obtained and have reproducibly created stable AtT-20 cells lines that express the antisense Zis-SR. Zis-SR mRNA expression was significantly reduced using different antisense constructs as compared to control AtT-20 cell lines (LacZ and vector-transfected controls were also prepared). Stable AtT-20 cell lines expressing antisense Zis-SR were analyzed by immunocytochemistry to determine if secretory granules had been affected (FIG. 12). Briefly, the cells were labeled with antibodies directed against carboxypeptidase E (CPE), and analyzed by light microscope (Fricker et al., 1993; Song et al., 1995). At the light microscope level, control AtT-20 cells were stained (wild type, LacZ-transfected, and vector-transfected) as well as two different AtT-20 cells lines with highly reduced Zis-SR expression (10-20% of normal levels) with antibodies directed to carboxypeptidase E (CPE). CPE is an enzyme that removes C-terminal charged amino acids (lysine or arginine) from peptides or proteins. CPE is also known to be an important component of secretory granules and thus can serve as an excellent marker for the presence of secretory granules. The CPE antibodies were directed to the N-terminal and the C-terminal regions of CPE and gave identical results. The antibodies were used to stain control AtT-20 and AtT-20 expressing antisense Zis-SR (AtT-20-As) cell lines. Detection was carried out using an anti-rabbit IgG second antibody conjugated to FITC. Wild type AtT-20 cells were heavily stained showing immunoreactivity in extensions and projections as expected. Staining was also observed in a perinuclear pattern within the Trans Golgi Network (TGN). However, AtT-20-As cells no longer showed any immunoreactivity in their-extensions, suggesting that granule formation had been severely compromised. Decreased CPE immunoreactivity was not due to CPE transcriptional/mRNA turnover effects or random genomic effects of the antisense Zis-SR transfection, since a Northern blot analysis of AtT-20 Zis-SR antisense cell lines demonstrated that CPE mRNA expression was identical to that of control AtT-20 cells (FIG. 13A). However, the intracellular content of CPE is clearly reduced as shown by Western blots of control AtT-20 cells compared to AtT-20 cells expressing the antisense constructs. The best clone, 700 As-3, contains significantly lower levels of CPE than that of AtT-20 cells. Since RNA levels are the same, this strongly suggests that the storage of CPE has been compromised. Functional tests of the regulated secretory capacity of the stable AtT-20 cell lines expressing antisense Zis-SR were conducted (FIG. 14). By Western blot analysis, CPE release following short term depolarization conditions (56 mM KCl) were examined. Control AtT-20 cells responded to depolarization with a large increase in CPE release observed in the media. However CPE levels in the media were not significantly increased in AtT-20 cells expressing antisense Zis-SR treated with identical depolarization conditions. These data demonstrate that the secretion of CPE is no longer “regulated” in the 700 As-3 clone, like in normal AtT-20 cells. An ultrastructural analysis of AtT-20 Zis-SR antisense cell lines also showed a significant reduction in dense core secretory granules (FIG. 15).

Taken together, the sum of these data demonstrate that Zis-SR expression is vital to the formation of secretory granules and the presence of a functional regulated secretory pathway.

The analysis of these clones demonstrates that endogenous Zis-SR expression is reduced by 80-90% as compared to wild type AtT-20 cells, or AtT-20 cells stably transfected with vector alone or with LacZ. Stable AtT-20 cell lines were analyzed.

The present invention is illustrated in further detail by the following non-limiting examples.

### EXAMPLE 1

### Reagents and Cell Culture

Culture media, hygromycin, G-418, trypsin and FBS used for cell culture was obtained from Gibco-BRL Life technologies. Rnase free Dnase was obtained from Promega. Restriction enzymes, RNA polymerases (SPC6 and T7), sequencing kits and ECL+kit for Western blots were obtained from Amersham-Pharmacia Biotech. T4 ligase was obtained from USB scientific. The vectors pcDNA3.1-hygro, pCRII-TOPO and TOP10 cells were obtained from Invitrogen. The CPE C-terminal antibody was obtained from Dr. L. Fricker (Albert Einstein University, Bronx, N.Y.). Anti-IgG antibodies coupled to FITC were purchased from Sigma. All electron microcopy materials were obtained from Electron Microscopy Sciences.

AtT-20 cells are corticotroph cell lines that were cultured in complete D-MEM (4.5 mg/L D-glucose) containing 10% inactivated FBS and 28 μg/ml gentamycine. For passages, the cells were washed with 4 ml of PBS (0.9% saline containing 2.7 mM KCl, 8.1 mM Na₂HPO₄, 1.15 mM KH₂PO₄) and then treated with a 2 ml of 0.05% trypsin in 1× Versene (137 mM NaCl, 2.7 mM KCl, 8 mM Na₂HPO₄, 1 mM KH₂PO₄, 0.5 mM EDTA). The cells were incubated for 5 min at 37° C. The cells are collected and centrifuged and then resuspended in 1/4 of the culture media for re-plating. The cells were maintained at 37° C. at 100% humidity and 5% v/v CO₂. The 6T3 cells originate from the fusion of the mouse myeloma 4T001 and AtT-20 cells. They are maintained in D-MEM (4.5 mg/L D-glucose) supplemented with 10% v/v inactivated FBS, containing 28 μg/ml gentamycin and 250 μg/ml G418. In order to differentiate 6T3 cells, the cells are first washed with PBS, followed by stimulation with 8-bromo-cyclic AMP (8-br-cAMP) at a final concentration of 5 mM in fresh culture media.

### EXAMPLE 2

### Antisense Constructions

Three different antisense constructions were tested. All included the initiator codon ATG of Zis-SR. The insert sizes were of 414, 737 and 1375 nts. These three fragments were amplified by PCR using distinct sets of oligos and the resulting DNA fragment obtained was subcloned into the pcDNA3.1-hygro vector. All three amplification reactions used the Zis5 primer (5′aggtctgctgctggcgtccaagatgtc3′). For the 414 fragment the RDF7 primer was used, for the 737 fragment the GP2-AS primer (5′ctggactgcgaactggaagagcttctt3′) was used and for the 1375 fragment the RDF5 primer was used (5′ggcaaccctgtctctgt 3′).

### EXAMPLE 3

### Cell Transfections

Cells were brought to 60-70% confluence, and washed in Opti-MEM™ Reduced Serum Media and transfections were carried out using LipofectAMINE™. For a 100 mm dish, 5 μg of plasmid DNA and 20 μl of LipofectAMINE™ were diluted in separate tubes in Opti-MEM™ to a volume of 1900 μl. The DNA solution is then added dropwise to the LipofectAMINE™ solution. Following a 45 min incubation, 800 μl of Opti-MEM™ is added and this solution is then added dropwise to the petri dish that contains 4 ml of Opti-MEM™. Following 6 hrs of incubation at 37° C., the cells are washed with 10 ml of complete media. After 24 hrs post-transfection, the cells were washed with PBS and 48 hrs is post-transfection, the cells were selected with 250 μg/ml of hygromycin. One week following hygromycin selection, cell death is observed by cells that have not incorporated the plasmid DNA. The cells are diluted 1:3 in 150 mm dishes. Selection is carried out for another 3-4 weeks. Single cell colonies are collected and propagated in 24 well plates. Cells are eventually brought to confluence and used for RNA extraction, immunocytochemistry, protein extraction or secretion experiments.

### EXAMPLE 4

### Immunostaining

AtT-20 cells were cultured in special chambers for immunocytochemistry (FLACON Culture slides). After 2 days, when the cells had adhered to the slides, they were washed 3×in PBS and then fixed with 4% buffered paraformaldehyde for 30 min. The cells were then washed with PBS and submitted to permeabilization treatment for 1 hr with 0.1% Triton X-100 and 2 mg/ml BSA in PBS. The cells were washed 3× with PBS and 0.1% Triton and the primary antibody was then applied. The carboxypeptidase E (CPE) antibody was obtained from Dr. L. Fricker (Albert Einstein University) and the chromogranin A (CgA) antibody was obtained from Dr. Sven-Ulrik Gorr (University of Louisville Health Sciences Center). The antibodies were incubated overnight at 4° C. This is followed by application of the secondary antibody after a 3×wash with PBS 0.1% Triton. The secondary antibody is incubated for 1-2 hrs at room temperature. The slides are mounted with antifade solution (2.5% DABCO, 200 mM Tris-Hcl pH 8.6, 90% glycerol).

### EXAMPLE 5

### Secretion Experiments

Cells were stimulated with a 4 ml solution of physiological Krebs solution containing 56 mM KCl for 30 min. Depolarization provokes a release of secretory granules and the short time period precludes the formation of novel proteins through a transcriptional effect. Control cells were treated with a similar 4 ml solution, however the KCl concentration was maintained a 4.7 mM. The media was collected after 30 min and concentrated on Centricons YM-10. With a molecular weight exclusion of 10,000 kDa.

### EXAMPLE 6

### Western Blots

Cells were extracted in extraction buffer containing protease inhibitors (50 mM Tris-HCl, 2.5 mM EDTA, 150 mM NaCl, 0.02% sodium azide, 2 μg/ml leupeptine, 2 μg/ml aprotinine, 2 mM P-mercaptoethanol, 100 μg/ml PMSF, 100 μM pepstatine). The cells were extracted using the freeze thaw method. Following centrifugation at 15,000×g for 30 min at 4° C., the supernatants were collected and tested for protein content using the Bradford assay. The protein samples were loaded on to 10% SDS-PAGE gels and following migration were transferred to Hybond™ P PVDF membranes using electrophoretic transfer. Primary antibodies to CPE or CgA were applied to the membranes overnight and detection was carried out using the ECL+ chemiluminescence kits (Amersham). The membranes are exposed with X-OMAT AR™ X-ray film (Eastman Kodak).

### EXAMPLE 7

### Electron Microscopy

Cell pellets were resuspended and fixed in a 5% gluteraldehyde in 0.1 M sodium cacodylate buffer (pH 7.4) in Krebs Hepes (1/1 v/v) for 20 min at 25° C. The cells were then pelleted and resuspended in 2.5% gluteraldehyde in 0.1 M sodium cacodylate buffer (pH 7.4) in Krebs Hepes (1/1 v/v) for 20 min at 25° C. The cells were once again pelleted and a solution of 1% osmium tetroxide in sodium cacodylatebuffer is added for 5 min. The solution was then replaced with a fresh solution of 1% osmium tetroxide and fixed for 1 h at 4° C. The pellets were carefully resuspended in a 0.25% uranyl acetate solution in 2% sodium acetate buffer (pH 6.3). The pellets were incubated for 45 min at 4° C. The pellets were then rinced overnight at 4° C. in a 0.25% uranyl acetate dissolved in water (pH 6.3). The fragments were re-suspended and dehydrated for 5 min in acetone solutions of 30, 50, 70, 80, 90 and 100% at 4° C. After a final dehydration step in 100% acetone, the fragments were placed in a epon-oxide propylene mixture (1/1) at 25° C. and then transferred to epon-oxide (4/1) overnight. The fragments were rinced in pure epon and then polymerised for 48 hrs at 60° C. Sections were stained with 1% uranyl acetate and 6% lead citrate (pH 10).

### EXAMPLE 8

### Northern Blots and RNA Extraction

Total RNA was extracted from cell lines using a guanidium isothiocyanate methodology, which includes a lithium chloride precipitation step (Day et al. 1995). The RNA (5 μg) was separated on agarose gel containing 6% formaldehyde and blotted on Nytran Plus™ membranes (Schleicher & Schuell, Keene, NH). Prehybridization was carried out for 2 h at 65° C. in hybridization buffer containing 5% SDS, 0.4 M sodium phosphate buffer, 1 mM EDTA, BSA 1 mg/ml, and 50% formamide. The purified ³²P-UTP-labeled cRNA probe was then added to the prehybridization buffer and incubated with the Nytran membrane overnight at 65° C. Filters were washed in 0.1×SSC, 0.1% SDS, 1 mM EDTA at 72° C. for 2 h and exposed to x-ray film (Kodak XAR-5 with intensifying screens) at −80° C. for 3 hours.

### EXAMPLE 9

### In Situ Hybridization

The in situ hybridization studies were carried out using antisense and sense labeled cRNA probes (³⁵S-UTP/³⁶S-CTP-labeled). Radioactive probes were diluted to 33×10³ dpm/μl and in situ hybridization was performed as previously described (Schafer et al., 1995). CD1 mice were sacrificed by rapid decapitation and tissues were rapidly removed and frozen in isopentane cooled to −35° C. The extracted tissues were stored at −80° C. until cryosectioning. Frozen 10 μm sections were cut on a Reichert cryostat™ (Leica Microsystems, Depew, N.Y.) and thaw-mounted on polylysine-coated glass slides and stored at −80° C. until processing. Sections were hybridized with Zis-SR cRNA probes. These sections were submitted to standard in situ hybridization procedure (Schafer et al., 1995) followed by x-ray film autoradiography for 1-7 days resulting in low resolution images. These slides were also dipped in NBT nuclear emulsion (Kodak) to obtained cellular resolution. The sections hybridized were counterstained with cresyl violet, cleared in xylene, and mounted with Permount™ histological mounting medium (Fisher Scientific). As a negative control, radioactively labeled sense-strand probes of the same size and specific activity were used instead of the antisense-strand probe.

Although the present invention has been described hereinabove by way of preferred embodiments thereof, it can be modified without departing from the spirit and nature of the subject invention as defined in the appended claims.

### REFERENCES

Achsel, T. & Shimura, Y. (1996) J Biochem (Tokyo). 120, 53-60.

Arranz, V., et al. (1997) Mol Cell Biol. 17, 2116-26.

Bauren, G., et al (1996) J Cell Biol. 133, 92941.

Blencowe, B. J., et al (1995) et al Rna. 1, 852-65.

Bourquin, J. P et al (1997 ) Nucleic Acids Res. 25, 2055-61.

Caceres, J. F., et al (1997 ) J. Cell Biol. 138, 225-38.

Caricasole, A., et al (1996 Proc Natl Acad Sci USA. 93, 7562-6.

Chabot, B. (1996) Trends Genet 12, 472-8.

Clemens, K. R et al (1993) Science. 260, 530-3.

Corden, J. L. & Patturajan, M. (1997 Trends Biochem Sci. 22, 413-6.

Crozat, A., et al. (1993) Nature. 363, 640-4.

Day et al., (1995), DNA and Cell Biology 14:175-188.

De Bie, I., et al. (1996) J. Cell Biol. 135, 1261-75

Du, K. & Taub, R. (1997) Gene. 204, 243-9.

Du, L. & Warren, S. L. (1997) J. Cell Biol. 136, 5-18.

Duncan, P. I., et al (1998) Exp Cell Res. 241, 300-8.

Duncan, P. I., et al. (1997) Mol Cell Biol. 17, 5996-6001.

Fetzer, S et al. (1997), Rna. 3, 344-55.

Finerty, P. J., et al (1997) J Mol Bol. 271, 195-208.

Frickeret al., 1993, J. Neurochem. 61:1404-15.

Fu et al., 1995, RNA 1:663-680.

Gallego, M. E., et al. (1997) Embo J. 16, 1772-84.

Gross, T., et al (1997) Nucleic Acids Res. 25, 1028-35.

Gui, J. F., et al (1994) Nature. 369, 678-82.

Gui, J. F., et al (1994) Proc Natl Acad Sci USA. 91, 10824-8.

Hanamura, A., et al (1998) Rna. 4, 43044.

Jumaa, H. & Nielsen, P. J. (1997) Embo J. 16, 5077-85.

Kanopka, A., et al (1998) Nature. 393, 185-7.

Karginova E A, et al. 1997 Am J Physiol 273:F731-F738

Kim, E., et al (1997) J. Cell Biol. 136, 19-28.

Ladomery et al., 2000@ , Gene 256:293-302.

Liang et al., 1998, Mol. Biotechnol. 3:261-7.

Matsuuchi et al., 1991, J. Cell. Biol. 112:843-852.

Manley et al., 1996, Genes Dev 10:1569-79.

Mayeda, et al (1992) Proc Natl Acad Sci USA. 89, 13014.

Misteli, T., et al (1998) J. Cell Biol. 143, 297-307.

Nayler, O., et al (1997) Biochem J. 326, 693-700.

Neugebauer, K. M., et al (1995) J. Cell Biol. 129, 899-908.

Sarkissian, M., et al (1996) J Biol. Chem. 271, 31106-14.

Schafer et al., 1995, Methods Neurosci. 23:1644.

Seidah et al., 1994, Biochimie 76:197-209.

Shi, Y. & Berg, J. M. (1995) Science. 268, 2824.

Song et al., 1995, J. Neurochem. 65:444-53.

Tian, M. & Maniatis, T. (1993) Cell. 74, 105-14.

Tronchere, H., et al (1997) Nature. 388, 397-400.

Wu, J. Y. & Maniatis, T. (1993) Cell. 75, 1061-70.

Xu, R. M., et al. (1997), Structure. 5, 559-70.

Yeakley et al., 1999, J. Cell. Biol. 145:447-55.

Zahler, A. M., et al (1993) Science. 260, 219-22.

What is claimed is:

1. An isolated nucleic acid molecule encoding a protein involved in the secretory pathway in a cell comprising an amino acid sequence as set forth in one of FIGS. 4, 5 or 6, or homolog or variant thereof.

2. The isolated nucleic acid of claim 1, wherein said nucleic acid molecule encodes a protein involved in the formation of secretory granules.

3. An isolated nucleic acid sequence which hybridizes under high stringency condition to the isolated nucleic acid sequence of claim 1 or 2.

4. An isolated polypeptide involved in the formation of secretory granules in cells comprising the amino acid sequence spanning amino acid 243 to amino acid 310 in FIG. 4.

5. The isolated polypeptide of claim 4, having a sequence as set forth in one of FIGS. 4, 5 or 6, fragments or variants thereof.

6. The isolated polypeptide of claim 4 or 5, encoded by the nucleic acid sequence set forth in FIG. 4.

7. Use of anyone of the polypeptide or nucleic acid molecule of claims 1 to 6, to restore or increase the secretory properties of a cell.

8. A method of restoring the neuroendocrine differentiation of a cell, comprising a use of the nucleic acid molecule or polypeptide of one of claims 1 to 6.

9. A method of identifying a gene and/or protein involved in inducing regulated secretion, comprising a comparison at the molecular level of:
a) a secretion-defective cell line under conditions which restore differentiation of said secretion-defective cell, such that secretion is restored; and
b) said secretion-defective cell line in the absence of said conditions.

10. The method of claim 9, wherein said cell line is 6T3 cells and said condition which restore reaction is cAMP or agents which affect the cAMP-related signaling pathway.

11. Method of modulating the secretory properties of a cell comprising modulating the activity and/or level of Zis-SR.

12. The method of claim 11, comprising an administration to said cell of an agent selected from an antisense, an antibody or an agent which modulates the phosphorylated state of said Zis-SR.

13. An assay to identify a modulator of regulated secretion in a cell comprising an assessment of a biological activity of Zis-SR, part or derivative thereof, in the presence of a candidate agent, wherein, a modulator of regulated secretion is selected when said biological activity of Zis-SR, part or derivative thereof, is measurably different in the presence of said candidate compound as compared to in the absence thereof.

14. The assay of claim 13, wherein said biological activity is selected from secretion and binding to a partner or part thereof.

15. The assay of claim 14, wherein said part thereof comprises the extended SR region of Zis-SR.

16. The assay of claim 14, wherein said Zis-SR further comprises the consensus sequence in accordance with the present invention.

17. The assay of claim 14, wherein said partner is selected from a peptide, a nucleic acid, a chemical entity or a chimera thereof.

18. The assay of one of claims 13 to 17, wherein said part thereof is comprised in a fusion protein.

19. The assay of one of claims 13 to 18, wherein said assay is an assay enabling high throughput screening of candidate agents.
